(19) United States
(12) Patent Application Publication
(10) Pub. No.: US 2014/0222040 A1
(43) Pub. Date: Aug. 7, 2014

(54) Method and Device for Connecting a Conduit to a Hollow Organ

(71) Applicants: Jin S. Park, Parsippany, NJ (US); Pramod Narayan Bonde, Woodbridge, CT (US)

(72) Inventors: Jin S. Park, Parsippany, NJ (US); Pramod Narayan Bonde, Woodbridge, CT (US)

(21) Appl. No.: 14/167,947

(22) Filed: Jan. 29, 2014

Related U.S. Application Data
(60) Provisional application No. 61/849,820, filed on Feb. 1, 2013.

(51) Int. Cl. A61B 17/00; A61B 17/11

(52) U.S. Cl. CPC A61B 17/0057; A61B 17/11
USPC 606/153; 29/446

(57) ABSTRACT

This invention provides an improved method for connecting a conduit to a hollow organ/structure and a unique device consisting of an expandable metallic mesh and a bio-compatible graft material.

### FIELD OF THE INVENTION

This invention relates to methods and device in general, and more particularly to surgical methods and device for connecting a conduit to a hollow organ/structure.

### BACKGROUND

Heart failure amounts for the largest area of spending by the Medicare amounting to 33 billion dollars a year. Heart disease remains one of the commonest diseases in the western world (1). One option for patients with end stage heart disease is transplant, but the limited donor availability makes this option extremely limited (approximately 2000 patients per year). Ventricular assist devices (VAD) have proven to be a reliable method of treating these patients by giving them survival benefit as well as good quality of life. These devices commonly attach between the left ventricle and transport blood to the ascending aorta to bypass the left ventricle. For supporting the right ventricle they are usually take blood from the right atrium and return it to the pulmonary artery thus bypassing and taking over the function of the right ventricle.

Although several technological modifications have been undertaken to improve on the reliability and improved functioning of these devices, specifically reducing the mechanical failure, the inflow cannula that connects the heart chambers to that of the VAD have remained unchanged for the large part. All the current devices use a tubular cannula made up of rigid material that protrudes within the lumen of the heart chamber.

This often leads to malposition, suction events due to collapse of chamber wall, abnormal eddy currents that can lead to thrombus formation and intermittent and sometime fatal pump dysfunction. Some design characteristics of the current cannula incorporate fenestrations or bevels at the tip or cages to prevent total obstruction of the cannula and optimize flow (2,3). Common problems with current cannulas have been well documented; intra-operative position of the cannula may be displaced following the closure of the patient necessitating design of flexible cannula with rigid tips (4-13).

Some design improvements have been reported to the cannula design but all of them require the chamber to be opened surgically and then inserting the cannulas, which is cumbersome and increases damage to the chamber (12-14). Additionally these are bulky devices unable to be flexible enough to allow minimally invasive approached to the placement of the cannula, which can be connected to the VAD device.

Several interventions within the heart structures and related blood vessels need an access and exit to the cardiac chambers. For this the cardiac apex is an ideal route allowing for a very short and direct route. For example, insertion of a stent mounted aortic valve, trans apical aortic or mitral valve replacement, atrial fibrillation ablation, insertion of a aortic stent graft or intervening on the coronaries can be done easily via the apex of the left ventricle. There is limited avenues for accessing and closing the heart apex at this stage, most of them involve inserting a titanium screw and cap, or surgical placement of sutures or polypropylene suture placement device.

Some prior art, in attempting to develop connector devices that implant in the heart wall, assumes a smooth heart wall of constant thickness and operates by sandwiching tissue between opposing parallel plates. See, for example, FIG. 12B of U.S. patent application Ser. No. 11/770,288, filed Jun. 28, 2007 by William E. Cohn for AUTOMATED SURGICAL CONNECTOR, and FIGS. SA and SB of U.S. patent application Ser. No. 11/251,100, filed Oct. 14, 2005 by Thomas Vassiliades eta. for VASCULAR CONDUIT DEVICE AND SYSTEM FOR IMPLANTING, which two patent applications are hereby incorporated herein by reference. In reality, however, the interior of the left ventricle of the heart is generally not a smooth continuous surface, and the wall thickness of the left ventricle generally varies considerably within any given patient, and also from patient to patient. As a result, the methods and apparatus disclosed in the aforementioned U.S. patent applications Ser. Nos. 11/770,288 and 11/251,100 can present issues when applied in actual patient anatomies.

Some references that discuss the requirements for successful implantation of an apico aortic conduit are listed below:
1. AHA annual statistics.
2. Holman, W. L., et al., Left atrial or ventricular cannulation beyond 30 days for a Thoratec ventricular assist device. ASAIO J, 1995. 41(3): p. M517-22.
3. Lohmann, D. P., et al., Left ventricular versus left atrial cannulation for the Thoratec ventricular assist device. ASAIO Trans, 1990. 36(3): p. M545-8.
4. Badiwala, M. V., H. J. Ross, and V. Rao, An unusual complication of support with a continuous-flow cardiac assist device. N Engl J Med, 2007. 357(9): p. 936-7.
5. Amin, D. V., et al., Induction of ventricular collapse by an axial flow blood pump. ASAIO J, 1998. 44(5): p. M685-90.
6. Reesink, K., et al., Suction due to left ventricular assist: implications for device control and management. Artif Organs, 2007. 31(7): p. 542-9.
7. Watanabe, K., et al., Development of a flexible inflow cannula with titanium inflow tip for the NEDO biventricular assist device. ASAIO J, 2004. 50(4): p. 381-6.
8. Hetzer, R., Proceedings of the 4th Berlin Symposium on Mechanical Circulatory Support. J Card Surg, 2006. 21: p. 512-520.
9. Snyder, Preclinical Biocompatibility Assessment of Cardiovascular Devices in Bioengineering in Bioengineering. 2006, University of Pittsburgh.
10. Miyake, Y., et al., Left ventricular mobile thrombus associated with ventricular assist device: diagnosis by transesophageal echocardiography. Circ J, 2004. 68(4): p. 383-4.
11. Votapka, T. V., et al., Left ventricular cannula obstruction in a patient with previous ventricular aneurysmectomy. Ann Thorac Surg, 1994. 58(4): p. 1182-4.
12. Griffith, B. P., et al., HeartMate II left ventricular assist system: from concept to first clinical use. Ann Thorac Surg, 2001. 71(3 Suppl): p. S116-20; discussion S114-6.
13. Vollkron, M., et al., Suction events during left ventricular support and ventricular arrhythmias. J Heart Lung Transplant, 2007. 26(8): p. 819-25.
14. Antaki, J. F., et al., An improved left ventricular cannula for chronic dynamic blood pump support. Artif Organs, 1995. 19(7): p. 671-5.
15. Curtis, A. S., et al., Novel ventricular apical cannula: in vitro evaluation using transparent, compliant ventricular casts. ASAIO J, 1998. 44(5): p. M691-5.
16. ASAIO Bioengineering/Tissue Engineering Abstracts. ASAIO Journal, 2007. 53(2): p. A1-69.

The present invention addresses the aforementioned difficulties associated with connecting an implantable connector to a hollow organ/structure.

### SUMMARY

A main object of the present invention is to provide a self-expanding muscular hollow organ connection device and a method of inserting such device. Specifically, the invention provides an improved access method and a cannula device that allows the improved access method. The proposed device design consists of an expandable metallic mesh and a bio-compatible graft material.

In one embodiment, the device consists of an expandable metallic mesh. The mesh can consist of a material selected from Co—Cr, Stainless Steel, and a shape memory material. In another embodiment, the shape memory material is nitinol or a nitinol alloy.

In another embodiment, the device has a first portion, a second portion, and a middle portion. The first and second portions can be located at opposite ends of the middle portion. In another embodiment, the first portion and second portion independently can contain one or more barbs at or near the edge of said portions for the purpose of self-anchoring the device once inserted into the hollow organ.

In another embodiment, the middle portion contains a bio-compatible graft material attached on the inside of the middle portion of the device. The graft material can be selected from one of Dacron, ePTFE and PTFE, polyester, polytetrafluroethylene, and collagen.

In yet another embodiment, the bio-compatible graft material can be selected from one of a polyester, polytetrafluoroethylene and collagen which is attached to the outside of the metallic mesh and located between the mesh and the organ tissue.

In another embodiment, the device is made of uni-body construction, cut from a nitinol tube. The process for making the device further comprises partially expanding a first portion and a second portion. Each portion is attached at opposite ends to a middle portion, and the first and second portions are thermally treated to form a flower shape.

In yet another embodiment, the device is made from a Co—Cr or stainless steel wire.

In one embodiment, the device is straightened, crimped and loaded into a small profile delivery system and thereafter deployed at the intended organ. Once deployed, the device regenerates back to its original expanded state. Upon expansion, the graft material attached to the inside of the middle portion of the device becomes a conduit that provides smooth access into and out of the hollow organ. The expanded device is further capable of spontaneous closure. When closed, the device provides a leak proof access point.

In one embodiment, the invention proposes a method of delivery of a transcutaneous or transapical aortic or mitral valve, manipulation of aortic or mitral valve or ablation of atrial fibrillation or insertion of a coronary stents or aortic stent grafts through the apex of the left or right ventricle or through a the wall of any cardiac chambers. The invention will allow a secure closure after the manipulating catheter or delivery system is removed.

In one embodiment, the invention provides a method of making a connection to a muscular hollow organ, facilitating entry and exit to said organ. In a further embodiment, the muscular hollow organ is the heart.

In another embodiment the device is a ventricular apical access device.

In yet another embodiment the method is an improved method for ventricular apical access to the heart. In a further embodiment, the ventricular apical access is to the left ventricle.

In yet another embodiment the method is an improved method for connecting the heart's vessels and chambers to the exterior by use of a Dacron, PTFE, polyester, nylon, or polypropylene tube material.

In another embodiment the device is used as a single multi-access site to a muscular hollow organ for surgical procedures. Some surgical procedures that may be improved by the use of the device of the present invention can be selected from the group consisting of ventricular apical access, percutaneous valve delivery, percutaneous gastrostomy, cystostomy, colostomy, ventriculoperotoneal shunt or any shunt procedures between blood vessels, and connection between hollow organs and exterior or to another hollow organ.

In yet another embodiment the procedure that may be improved by the use of the device is a post-operative procedure such as for example, support to the failing heart chambers.

The invention, as represented in one or more embodiments, has many advantages including but not limited to the following:
1. The device can be straightened and crimped to fit into a delivery device and then self-expands once deployed, thus making the procedure minimally invasive.
2. The device provides an improved connection to a hollow organ allowing spontaneous flow shut-off.
3. After implantation of the device into the wall of a hollow organ, a medical device can be easily delivered.
4. The device can provide a single multi-access site due to the super-elasticity of the nitinol frame, and
5. After implantation, the self-expanding and self-closing device can be secured to the heart without suturing.

Other details, objects, and advantages of the invention will become apparent from the following detailed description and the accompanying drawings and figures of certain embodiments thereof.

### BRIEF DESCRIPTION OF THE DRAWINGS

FIG. 1 is a top view of a self-expanding muscular hollow organ connection device in accordance with the present invention.

FIG. 2 is a side view of a self-expanding muscular hollow organ connection device in accordance with the present invention.

FIG. 3 is a cut tube prior to shape-setting.

FIG. 4 is a top view and a side view of a self-expanding muscular hollow organ connection device expanded for cannulation in accordance with the present invention.

FIG. 5 is a schematic view showing the deployment of the device in FIGS. 1, 2 and 4 into the left ventricle of a heart. The device is in the normally closed configuration.

FIG. 6 is a schematic view showing the opening of the device in FIGS. 1, 2 and 4 once inserted into the left ventricle of the heart, for the purpose of cannula insertion.

FIG. 7 shows the top and side viewss of a self-expanding muscular hollow organ connection device in the “normally closed” position and the “pushed open” position, and a schematic of the device in both positions once inserted into the hollow organ in accordance with the present invention.

FIG. 8 is a schematic showing the deployment of a muscular hollow organ connection device, the self-expanding mechanism of the nitinol frame to the normally open configuration and the graft material creating a conduit allowing for multiple surgical procedures in accordance with the present invention. Also shown is the placement of a clip on the graft material lining the inside of the frame that seals the opening of the access point.

### DETAILED DESCRIPTION

Definitions:

For the purposes of the present disclosure, the following terms shall have the associated meanings. Reference in any given embodiment to a term defined below is to be understood as incorporating the broadest definition of such term.

The term “cannula” shall mean a tube which can be permeable, impermeable, partially permeable, partially impermeable, or selectively permeable to fluid.

The term “stent” shall mean a structure that can support an anatomical structure, such as, but not limited to, a blood vessel, intestine or other structure, by exerting a force counter to a collapsing or shrinking force exerted by the anatomical structure.

The term “conduit” shall mean a fluid impermeable tube capable of conducting a fluid from a first location to a second location.

The terms “ePTFE” and “PTFE” shall mean expanded polytetrafluorethylene and polytetrafluorethylene respectively.

### DETAILED DESCRIPTION OF THE EMBODIMENTS

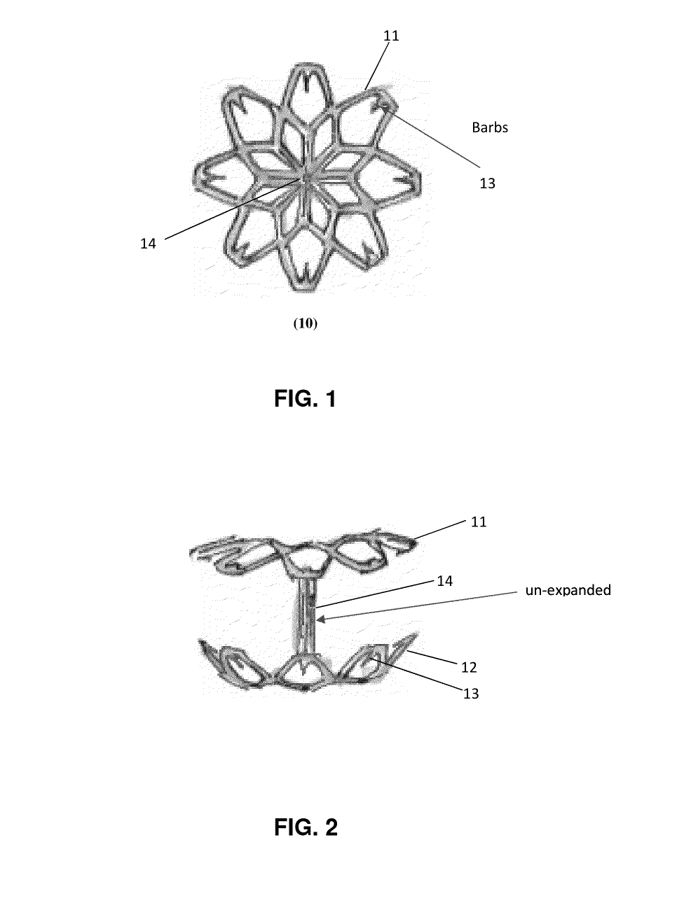

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the apparatus, system, and method of the present invention, as represented in FIGS. 1 through 8, is not intended to limit the scope of the invention, as claimed, but is merely representative of presently preferred embodiments of the invention.

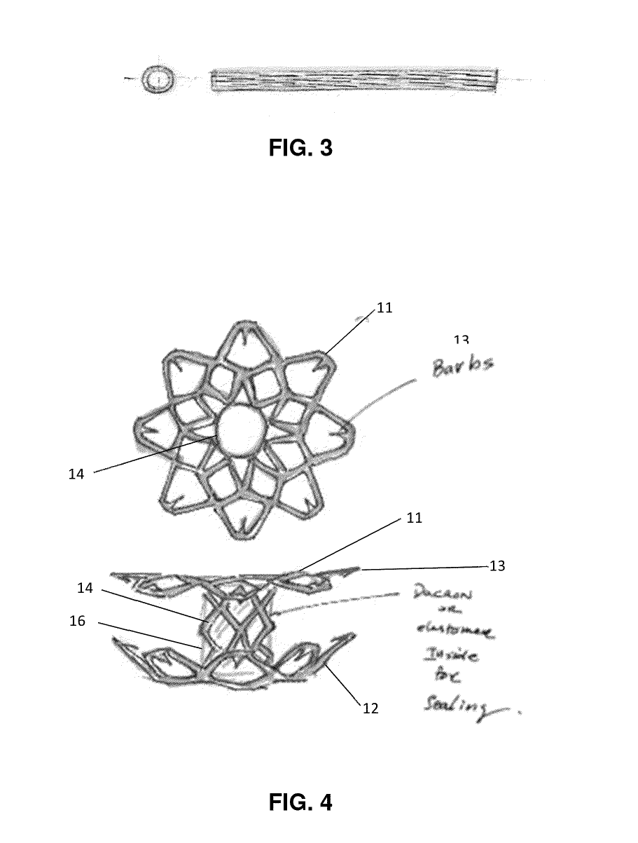

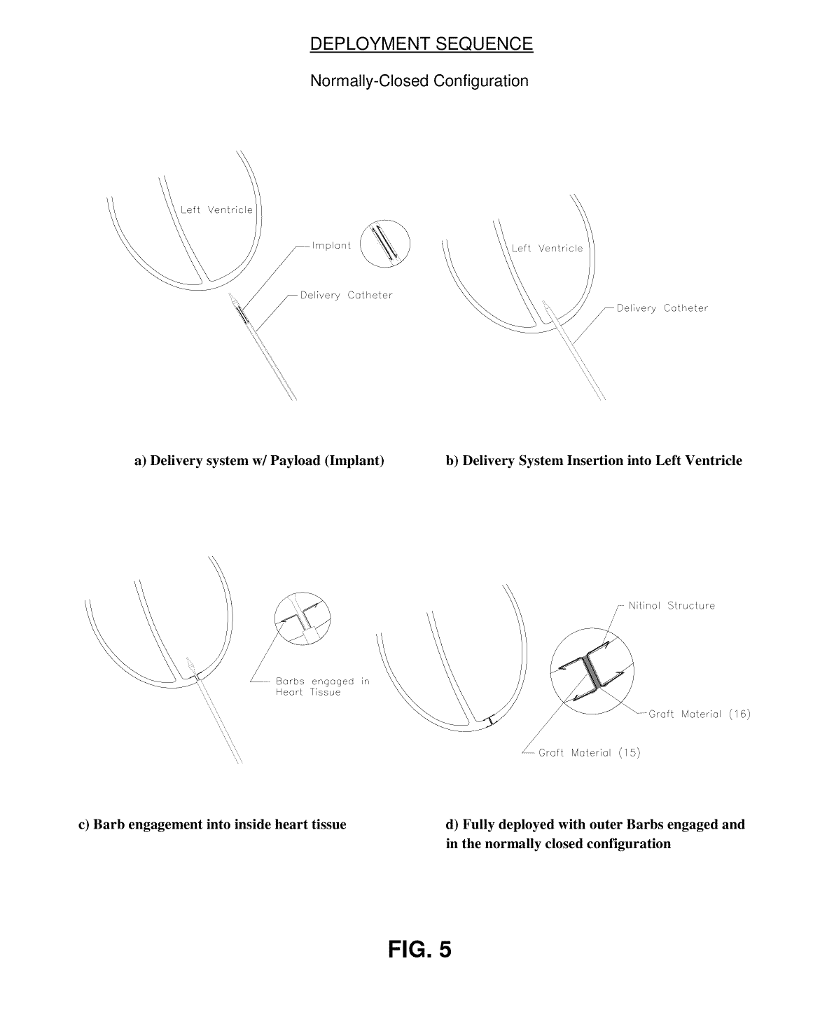

As illustrated in FIGS. 1 and 2, a device (10) for use in connecting to a hollow organ, is comprised of an expandable metallic mesh. The device has a first top portion (11), a second bottom portion (12), and a middle portion (14). The top (11) and bottom (12) portions are located at opposite ends of the middle portion (14). The device configuration in FIGS. 1 and 2 is cut from a nitinol tube (FIG. 3) and the top and bottom portions are partially expanded and thermally treated to form a flower shape. The metallic mesh material is a super elastic nitinol material which is super-elastic at body temperature.

Once inserted into the hollow organ, the top and bottom portions are in contact with an inner and outer wall respectively. The device (10) further independently comprises one or more barbs (13) at or near the edge of each of the first top portions and second bottom portions to enable self-anchoring of the device.

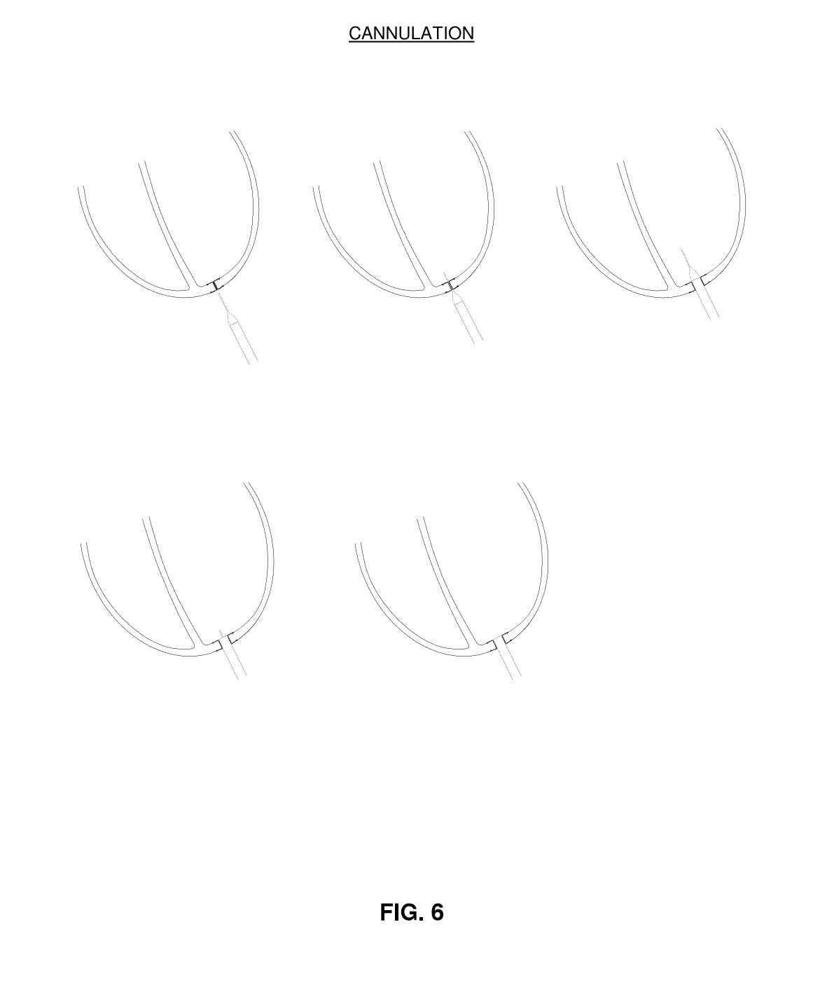

One unique aspect of the device (10) according to the present invention is its ability to self-expand (FIG. 4) once deployed into the hollow organ, for cannulation. In a preferred embodiment, the device (10) is deployed to the left ventricle of the heart by means of a small profile delivery catheter. Once inserted the super-elastic nitinol frame reverts to its normal open configuration (FIG. 4). The frame anchors itself to the inner and outer walls of the ventricle using barbs (13). The device (10) further comprises a bio-compatible graft material (16) located on the inside of the middle portion (14) of the frame, such as, for example, Dacron, ePTFE, PTFE and polyester. When opened for cannulation as shown in FIG. 4, the graft material becomes a conduit that provides smooth access to the ventricle. When deployed closed, the frame and the graft material maintain a leak-proof environment.

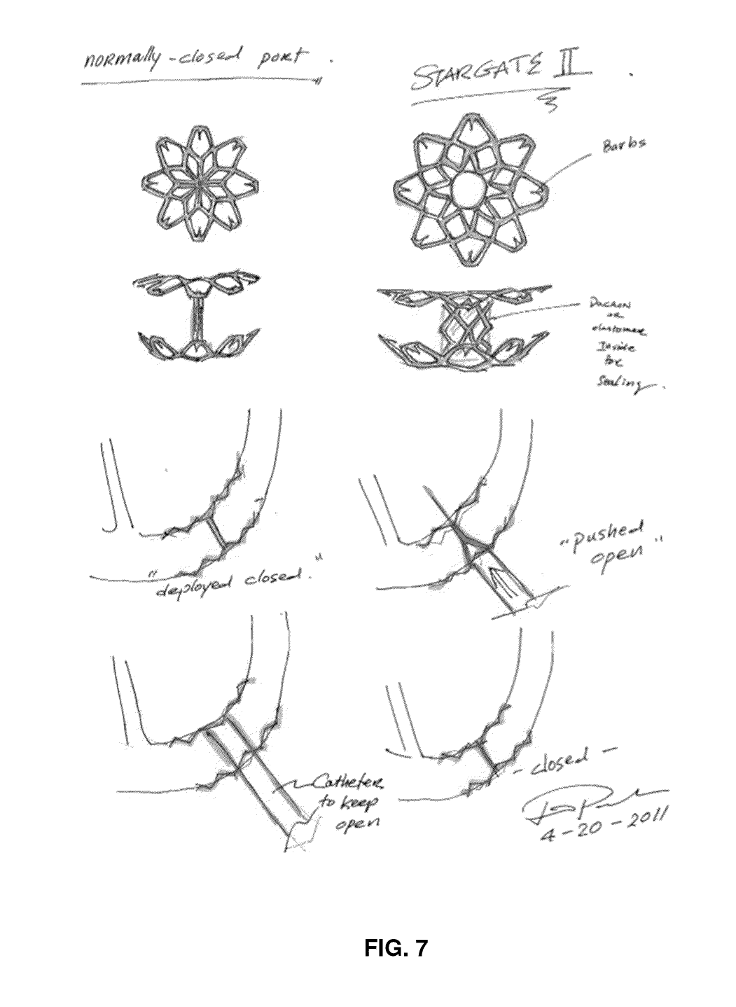

As illustrated in FIG. 5 the deployment sequence of the device (10) into the left ventricle of a heart, shows the delivery catheter having the device (payload) therein entering the left ventricle of the heart. Once contacting the inner wall of the ventricle, the first or top portion (11) of the device self-anchors into the inner wall by means of the barbs. Additionally, the bottom or second portion (12) of the device (10) engages with the outer wall and self-anchors thereto by means of the barbs.

In a particular embodiment, the barbs grab the heart tissues for stability. By capturing the muscle tissues inside and outside the ventricle during deployment, the device (10) moves along with the surrounding tissue and provides securement.

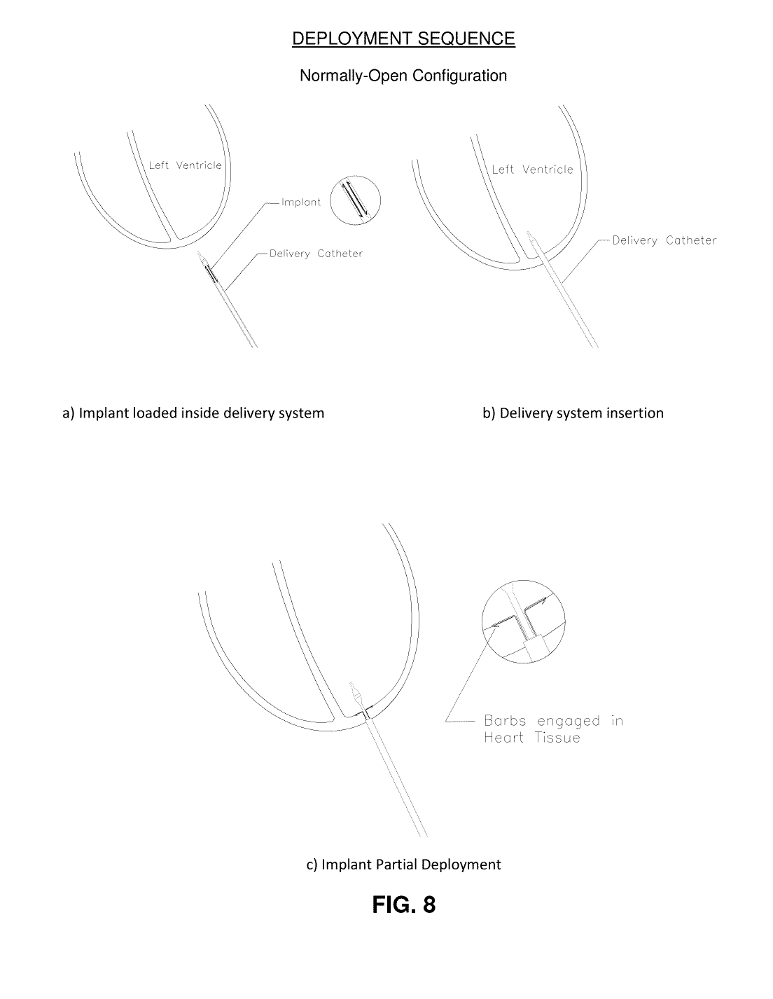

In a specific embodiment, the schematic view in FIG. 6 illustrates the opening of the device (10) of FIGS. 1, 2 and 4 once inserted into the left ventricle of the heart, for the purpose of cannula insertion.

This improved method of using the device (10) as depicted in FIG. 6, allows the site to be accessed multiple times for post-op procedures or other surgical procedures. Some procedures capable of being performed incorporating the use of device (10) of the present invention can be but are not limited to ventricular apical access, percutaneous valve delivery, percutaneous gastrostomy, cystostomy, colostomy, ventriculoperotoneal shunt or any shunt procedures between blood vessels, and connection between hollow organs and exterior, or to another hollow organ and the like.

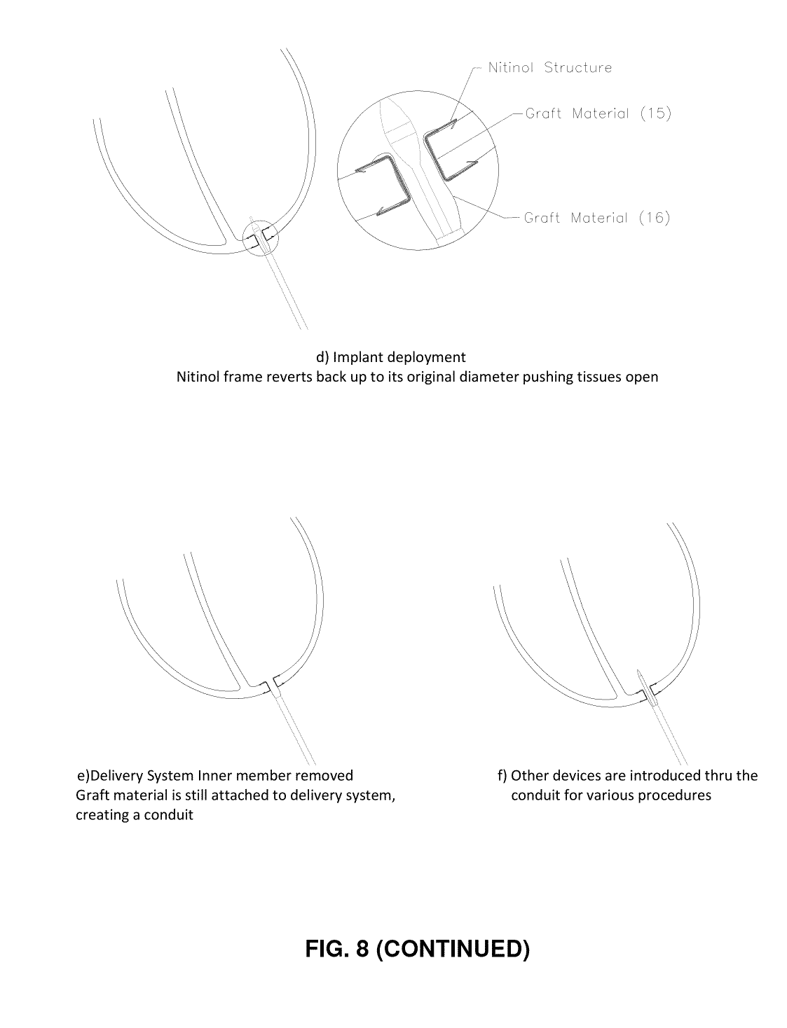

As depicted in FIG. 7 the top and side views of a self-expanding hollow organ connection device (10) is shown in the “normally closed” position and the “pushed open” position. In addition, a schematic illustrates the device in both positions once inserted into a hollow organ in accordance with the methods of the present invention.

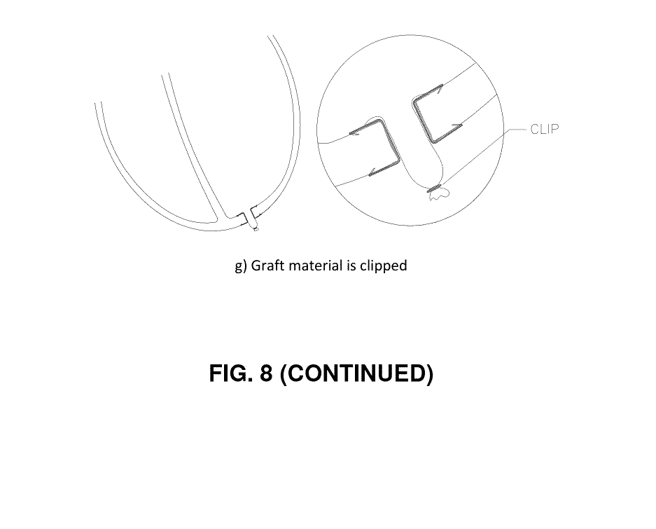

The present invention describes the method of deployment, schematically shown in FIG. 8, of a hollow organ connection device, the self-expanding mechanism of the nitinol frame to the normally open configuration and the graft material creating a conduit allowing for multiple surgical procedures in accordance with the present invention. In a further embodiment a clip is placed on the outwardly extending end of the graft material lining the inside of the frame, extending from the outer wall of the organ, which seals the opening of the access point.

This invention describes a new improved method of providing a self-expanding hollow organ connection device to a hollow organ. In one preferred embodiment, the organ is a muscular hollow organ, such as for example, the left ventricle of a human heart. This invention could be useful in other organs in a human or animal such as the right ventricle, the left or right atrium, the stomach, the bladder, blood vessels or other fluid filled organs.

Generally described, the invention consists of a self-expanding hollow organ connection device (10) consisting of a metallic mesh material and a bio-compatible graft material. The metallic mesh material forms a frame consisting of a top portion (11) a bottom portion (12) and a middle portion (14). The metallic mesh material useful in the methods of the present invention includes, but is not limited to, substances biologically inert and capable of forming a structure or with some degree of elastic properties. A wide range of materials including, but not limited to, metals, such as, but not limited to stainless steel and silver, nitinol, co—cr alloy, plastics, monofilament or multifilament polymer, shape memory polymers, or biological tissues or the like and/or mixtures, combinations, alloys or composites thereof, may be suitable.

In one embodiment the shape memory material is nitinol or a nitinol alloy material. Nitinol is a nickel-titanium alloy and probably the best known representative of the shape-memory alloys. Nitinol has a cubic crystal structure which comprises approximately 55 wt. % nickel and the remainder titanium. The alloy is usable up to 650° C., is corrosion resistant, and is very strong. The alloy is pseudo-elastically deformable up to approximately 8%. Shape-memory alloys, are well known in the art, in particular, nitinol, are used in medical technology in the form of, inter alia, self-expanding stents. A stent is a medical implant which is introduced into specific organs to support their walls all the way around. The nitinol stent is a small tubular support structure comprising nitinol, which may assume a compressed state having a small diameter and an expanded state having an enlarged diameter predefinable for the intended purpose.

The bio-compatible graft material useful in the present invention can be attached to either the inside, outside or both sides of the metallic mesh frame. For purposes of the present invention, the graft material when attached to the inside of the metallic mesh frame serves as a conduit allowing smooth access into and out of the hollow organ. Suitable bio-compatible graft materials for attachment on the inside of the mesh, include but are not limited to, Dacron, ePTFE, PTFE, and polyester. When attached to the outside of the frame, the graft material is placed between the frame and the organ tissue allowing the tissue to grow or fuse with the frame. Suitable graft materials for placement between the frame and the tissue include but are not limited to, Dacron, polyester, and collagen.

In one aspect of the invention, the device (10) can be used as a single multi-access site to a hollow organ for surgical procedures. Such procedures can be selected from but not limited to ventricular apical access, percutaneous valve delivery, aortic valve repair, mitral valve repair, PFO (Patent Foramen Ovale), percutaneous gastrostomy, cystostomy, colostomy, ventriculoperotoneal shunt or any shunt procedures between blood vessels, and connection between hollow organs and exterior or to another hollow organ.

Furthermore, the device (10) can be used as a single multi-access site to a hollow organ for post-operative procedures such as for example, support to the failing heart chambers.

In accordance with the above description, the present invention describes a new improved method of providing a self-expanding hollow organ connection device to a hollow organ. In one embodiment, the device (10) is loaded into a catheter and delivered to the hollow organ with minimal invasion. Upon first deployment of the device (10) at the organ site, the barbs on the first or top portion (11) of the device engage the inner wall. Upon complete deployment, the barbs on the second or bottom portion (12) engage the outer wall of the organ. For the purpose of the present invention, the barbs capture the tissues inside and outside during deployment, thereby allowing the device to move along with the surrounding tissue and provide securement of the connection device (10). The construction and composition of the device (10) allows it to be straightened, crimped and loaded in a small profile delivery system such as for example, a catheter. Upon deployment, the device composed of, for example, nitinol or a nitinol alloy material, resumes its original shape due to the super-elastic nature of the shape memory material, thereby forming the improved connection to the hollow organ.

As described earlier, the improved connection to a hollow organ provided according to the methods of the present invention, can serve as a multi-access point for surgical and post-surgical procedures on hollow organs thereby limiting the need for additional access points and reducing the stress and trauma often associated with such invasive procedures.

While the above description focuses primarily on attachment of a connection device to a hollow organ, such as a heart, it should be understood that the same device and procedures will allow attachment of the devices to other hollow organs, for example but not limited to gastrointestinal and urinary organs (i.e. for electrical stimulation and or monitoring of the GI tract), access to the bladder for enhancement of function or treatment of disease such as bladder cancer, implantation of apparatus such as stomach bypass tubes for treatment of morbid obesity or for limiting passage through the pylorus valve, access for implanting augmentation or enhancement devices for closure of body lumens such as magnetic or mechanical sphincters, endoscopic delivery means for diagnosing/treating gastric disorders, delivery of a resident sensing device, therapeutic delivery device, access means for removing tumors from hollow organs, access means for delivering and removing tumor treatment devices (i.e. radiation devices), access means for attaching graft to blood vessels, means of simultaneous cut-and-attach graft, and the like.

Although the invention has been described in terms of particular embodiments and applications, one of ordinary skill in the art, in light of this teaching, can generate additional embodiments and modifications without departing from the spirit of or exceeding the scope of the claimed invention.

Accordingly, it is to be understood that the drawings and descriptions herein are proffered by way of example to facilitate comprehension of the invention and should not be construed to limit the scope thereof.

What is claimed is:

1. A self-expanding hollow organ connection device (10) consisting of a metallic mesh material and a bio-compatible graft material wherein said device has a first portion (11), a second portion (12), and a middle portion (14), wherein said first (11) and second (12) portions are located at opposite ends of said middle portion (14), and wherein a first portion is in contact with a first inner wall of said organ and a second portion is in contact with a second outer wall of said organ.

2. The device of claim 1 wherein the first portion and second portion independently contain one or more barbs (13) at or near the edge of said portions.

3. The device of claim 1 wherein the device is self-anchoring.

4. The device of claim 1 wherein the metallic mesh material is a bio-compatible metal material.

5. The device of claim 4 wherein the metallic mesh material is selected from the group consisting of Co—Cr, stainless steel and silver, nitinol, plastics, monofilament or multifilament polymer, shape memory polymers, and biological tissues and mixtures, combinations, alloys and composites thereof.

6. The device of claim 1 wherein the metallic mesh material consists essentially of a shape memory material.

7. The device of claim 6 wherein the shape memory material is selected from the group consisting of a super-elastic nitinol and a super-elastic nitinol alloy.

8. The device of claim 1 wherein said bio-compatible graft material (16) is attached to the inside of the middle portion (14) of the metallic mesh.

9. The device of claim 1 further comprising a second bio-compatible graft material (15) attached to the outside of the metallic mesh.

10. The device of claim 8 wherein the bio-compatible graft material (16) is selected from the group consisting of Dacron, ePTFE, polytetrafluorethylene, and polyester.

11. The device of claim 9 wherein the second bio-compatible graft material (15) attached to the outside of the metallic mesh is located between the mesh and the organ tissue and is selected from the group consisting of Dacron, polyester and collagen.

12. The device of claim 1 wherein said device is a uni-body construction.

13. The device of claim 1 wherein said device is leak-proof when not expanded.

14. The device of claim 1 wherein said device is capable of being straightened, crimped and loaded into a small profile delivery system.

15. The device of claim 1 wherein the device is a ventricular apical access device.

16. A method of making a connection to a hollow organ, facilitating entry and exit to said organ comprising:
a. providing the device (10) of claim 1;
b. straightening and crimping said device (10), and loading it into a small profile delivery system, and
c. deploying said device (10) in the hollow organ.

17. The method of claim 16 further comprising, after deployment of the device (10), said device containing the graft material on the inside of the middle portion (14) self-expands and remains in the open position, thereby providing a conduit for smooth access.

18. The method of claim 17, further comprising, the middle portion (14) of device (10) of claim 1, spontaneously returning to a non-expanded state, thereby making the connection leak-proof.

19. The method of claim 16, wherein the hollow organ is the heart.

20. The method of claim 16 wherein, the method is an improved method for ventricular apical access to a hollow organ, wherein the organ is the heart and wherein the ventricular apical access is to the left ventricle.

21. The use of the device (10) of claim 1 as a single multi-access site to a hollow organ for surgical procedure.

22. The use according to claim 21 wherein, the surgical procedure is selected from the group consisting of ventricular apical access, percutaneous valve delivery, aortic valve repair, mitral valve repair, PFO (Patent Foramen Ovale), percutaneous gastrostomy, cystostomy, colostomy, ventriculoperotoneal shunt or any shunt procedures between blood vessels, and connection between hollow organs and exterior or to another hollow organ.

23. A method of making the device (10) of claim 1 wherein the device is cut from a nitinol tube, said method further comprising,
a. partially expanding a first portion (11) and a second portion (12) attached at opposite ends to a middle portion (14), and
b. thermally treating said first and second portions to form a flower shape.

24. A method of making the device (10) of claim 1 where in the device is made from a bio-compatible metallic wire selected from the group consisting of Nitinol, Co—Cr, and Stainless Steel.
